(19) United States
(12) Patent Application Publication
(10) Pub. No.: US 2011/0164721 A1
(43) Pub. Date: Jul. 7, 2011

(54) X-RAY IMAGE RECORDING SYSTEM AND X-RAY RECORDING METHOD FOR RECORDING IMAGE DATA WITH X-RAY UNITS FOR VOLUME RECONSTRUCTION

(75) Inventors: Emanuel Jank, Berlin (DE); Eckart Uhlmann, Kiebitzreihe (DE)

(73) Assignee: Fraunhofer-Gesellschaft Zur Forderung Der Angewandten Forschung e.V., Munich (DE)

(21) Appl. No.: 13/009,994

(22) Filed: Jan. 20, 2011

Related U.S. Application Data
(63) Continuation of application No. PCT/EP2009/005437, filed on Jul. 27, 2009.

(30) Foreign Application Priority Data
Jul. 31, 2008 (DE) 102008035736.7

(51) Int. Cl. A61B 6/03

(52) U.S. Cl. 378/4

(57) ABSTRACT

The present invention relates to an x-ray image recording system for recording x-ray projection images of alignment information for recorded x-ray projection images, comprising an x-ray tube and an x-ray image detector being arranged in the optical path of the x-ray tube for recording x-ray projection images of an object that can be disposed and/or that is disposed between the x-ray tube and the x-ray detector in an imaging region in a locally fixed manner, wherein the x-ray tube and the x-ray detector are disposed in a locally fixed manner relative to each other, and can be moved about the imaging region, at least in sections, a position sensor being disposed relative to the x-ray tube and the x-ray detector in a locally fixed manner, by means of said sensor the current alignment of the x-ray tube and the x-ray detector can be determined relative to a predefined reference direction at the moment of recording an x-ray projection image, and a storage unit for storing recorded x-ray projection images together with the respective current alignment of the x-ray tube and the x-ray detector. The invention comprises a computer being connected to the storage unit for the purpose of data exchange, by means of said computer the position data of the x-ray projection image that are necessary for the calculation of the layered images if the reconstruction can be calculated from the stored, associated current alignment for the purpose of layered image reconstruction based on multiple recorded x-ray projection images for each recorded x-ray projection image utilized.

### PRIORITY INFORMATION

The present application is a continuation of PCT Application No. PCT/EP 2009/005437, filed on Jul. 27, 2009, that claims priority to German Application No. 102008035736.7, mailed on Jul. 31, 2008. Both applications are incorporated herein by reference in their entireties.

### BACKGROUND OF THE INVENTION

The present invention relates to an X-ray image recording system (and also to a corresponding X-ray image recording method) for recording X-ray projection images and for recording orientation information for the recorded X-ray projection images. The recording system or the recording method can thereby be achieved in particular within the scope of a commercially available C-arm system which is then configured with suitable hardware- and/or software measures for operation as X-ray image recording system according to the invention.

In medicine, imaging serves for displaying inner regions of a patient and for diagnosis and for checking treatment. During a surgical intervention, imaging by means of X-ray systems (generally C-arms) is widespread. In this type of imaging, X-rays penetrate the tissue to be imaged and are thereby weakened. A projection image of the radiographed object in which spatial information is displayed in a superimposed manner is produced on the image detector of the X-ray system. The information content of a projection image, in contrast to three-dimensional volume image data (subsequently also termed tomographic image data of a tomographic image reconstruction) is restricted. Exact checking of implant positions or assessment of repositioned joint surfaces after fractures is scarcely possible with projection images.

The technical requirement for reconstructing volume image data from projection images resides in determination of the position information and projection geometry required for this purpose. Furthermore, a plurality of projection images must be recorded from different spatial directions for a reconstruction of a tomographic image. It must thereby be taken into account that the object to be reconstructed is always imaged in the X-ray images (i.e. that the imaging region in which the object to be imaged is positioned is always imaged on this sensitive surface of the X-ray detector).

2D X-ray units (in particular C-arms) are used to record X-ray image data in the operating theatre. C-arms consist of a C-shaped recording unit on an adjustable and moveable mounting. On the ends of the C or C-arm, the X-ray source (X-ray tube) and the projector are mounted. The C can be positioned on an operating table such that the table with the patient is situated within the C, between X-ray source and detector, and hence in the optical path of the unit and an X-ray image of the patient can be recorded. C-arms offer the possibility of rotating the recording unit about the patient in a plurality of rotational directions and of recording projection images from different directions. A property of the C-arms is thereby that the central beam of the X-ray system generally does not extend through the axis of rotation. This construction allows significantly smaller and lighter mechanical constructions but has the result that, during a rotation of the C, the object moves out of the image centre.

In the state of the art, such C-arms for recording 3D image data have been modified by equipping the individual moveable axles of the C-arm with measuring means and motors in order to move the recording unit (tube and detector) on a path about the object to be recorded, thereby recording X-ray images and determining the position of the images in order to be able to reconstruct 3D image data therefrom. For example, the system Ziehm Vario 3D is known from the state of the art. This 3D C-arm is based on a standard C-arm mechanical unit which is equipped with additional encoders and motors. The system offers automatic movement of the C about the patient with automatic image recording. In order to keep the object in the image centre, the horizontal and vertical axes of the system are readjusted parallel to the C-movement. The rotation is effected about 135 degrees and subsequently offers a volume reconstruction.

The systems known from the state of the art have the disadvantage in particular that a plurality of sensors and motors and possibly a device for automatic orientation of the X-ray system must be integrated in a fixed manner in the C-arm. The mechanical complexity for such a design of an X-ray system is hence expensive.

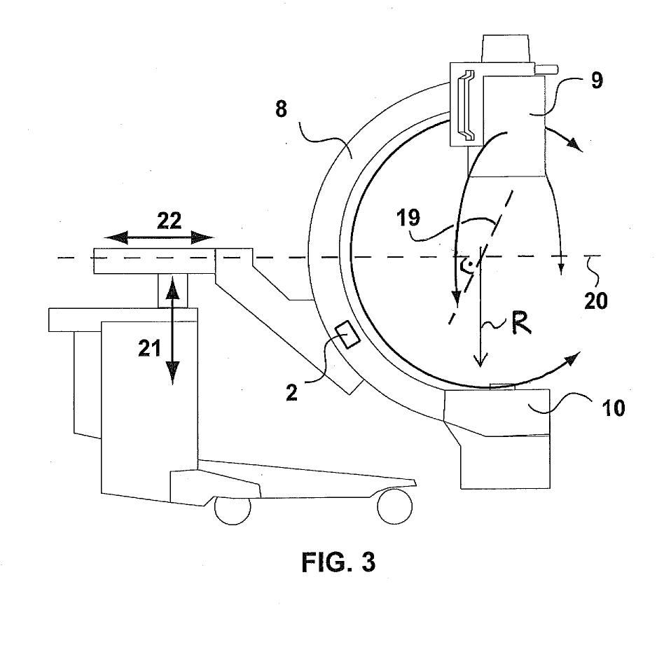

Furthermore, with the known devices, the freedom of movement in the 3D recording mode is restricted to a rotational direction (C-axis 19 or propeller axis or P-axis 20, see e.g. FIG. 3). As a result, the flexibility of the X-ray system is partially lost. The image recording cannot thus be adapted flexibly to the clinical problem and the desired reconstruction quality.

Finally, the known systems offer no possibility of improving the reconstruction outcome by specific recording of further images. After conclusion of the image recording and observation of the reconstruction outcome, it is not detectable from which directions further images should be recorded in order to improve the quality of the 3D reconstruction.

### SUMMARY OF THE INVENTION

It is hence the object of the present invention to make available an X-ray image recording system and an X-ray image recording method with which, in a simple, economical manner and with a simple mechanical construction (in particular the mechanical construction of a standard C-arm system), X-ray projection images of an object can be recorded from different directions and with which, by determining the position of the individual projection images in space (at the moment of their recording), all the required data (position data) for reconstruction of tomographic images from the recorded X-ray projection images can be determined with high precision.

This object is achieved by an X-ray image recording system according to patent claim 1 and also by an X-ray image recording method according to patent claim 16. Advantageous embodiments of the recording system or recording method according to the invention can be deduced respectively from the dependent claims.

Subsequently, a recording system (and hence also recording method) according to the invention is described firstly in general. Following thereon is a detailed concrete embodiment for the production of the recording system according to the invention.

The individual features of the special embodiment need not thereby be produced in the illustrated combination but can be produced also in any other combinations within the scope of the present invention.

It is the basic idea of the present invention to detect or to calculate those position data of each X-ray projection image which is used for image reconstruction of tomographic images not via a plurality of sensors/motors which are integrated in the recording unit in a fixed manner, but rather to derive these required position data on the basis of using a single position sensor. This position sensor (as described subsequently, it can thereby concern for example a sensor which determines the position of the recording system relative to the acceleration vector of gravitational acceleration) determines, for each projection image used for the reconstruction, the momentary orientation of the system comprising X-ray tube and X-ray detector at the moment of the recording of this projection image relative to a reference direction (i.e. for example the direction of gravitational acceleration).

For each recorded projection image used for subsequent image reconstruction, the associated momentary orientation of X-ray tube and X-ray detector, detected by the position sensor, is stored at the moment of the recording of the projection image together with the respective X-ray projection image so that an unequivocal assignment of orientation and X-ray projection image is provided here. As also described subsequently in detail, the required position data for each projection image used for the reconstruction are calculated from the stored, associated orientation. The position data are thereby those data which describe the position of the recorded projection image and the position of the X-ray tube at this moment in space such that, with reference to these data and the associated X-ray projection image, a tomographic image reconstruction with sufficient precision is possible. The calibration and the required position data are subsequently described in more detail (these are in detail the position/orientation of the image coordinate system relative to a basic coordinate system BKS (immoveable during the application/calibration), the scaling of the image (size of an image point) and the position of the X-ray source relative to the BKS or to the image.)

The conversion from orientations determined with the help of the position sensor into the required position data can take place for example in a computer of the system. However, it is likewise also conceivable that the stored X-ray projection images together with associated momentary orientations are transmitted, e.g. with the help of a portable hard disc, to an external computer system (PC or the like).

As is described likewise in more detail subsequently, the conversion or transformation of orientation data into required position data thereby takes place particularly preferably with the help of a pre-calibration of the system. In the case of such a calibration, the associated orientations can be detected, on the one hand, for various positions of the tube detector system with the help of the position sensor and, on the other hand, determination of the associated required position data can be undertaken with the help of an external calibration unit. The specific correlation between required position data and orientation data or orientation can be stored for example in the form of a look-up-table (LUT) in a memory so that, during operation of the recording system (and after removal of the external calibration unit), specific orientation values can then subsequently be converted into the associated required position data unequivocally (or almost unequivocally) with the help of the LUT.

Such a calibration unit can have for example a position measuring system (e.g. position camera), with which a three-dimensional calibration body, which is fitted in a fixed manner on the X-ray detector, can be evaluated with respect to its position and orientation in space (for example optically with subsequently connected image processing). Since the calibration body is disposed rigidly on the detector, conclusions can be made from determination of the position/orientation of the calibration body unequivocally with respect to the position and orientation of the detector (and hence with respect to the position of the tube-detector system). The thus obtained position data with respect to the position of the tube detector system are then stored with the simultaneously detected orientation data of the position sensor, as described above, in the form of a calibration table or LUT. During the actual recording operation (in which then a calibration system is no longer present but only the calibration table is still situated in the memory), the associated orientation can then be determined with the position sensor for each projection image to be used for the image reconstruction at the moment of its recording and, for example with the help of an interpolation method, the associated required position data can be determined from the LUT storing corresponding support points.

A particular advantage of the definition according to the invention of precisely one reference direction (to which the orientation data relate) is that, with the help of a single position sensor, which can also possibly be fitted subsequently, all the required data (e.g. during the above-described calibration) can be detected with high accuracy.

In a further advantageous embodiment, the system according to the invention has a reference unit, with which, on the basis of the associated orientations of already recorded X-ray projection images, further orientations of the X-ray tube-X-ray detector system can be calculated by means of suitable algorithms of the system and can be output, at which also further X-ray projection images must be recorded for an optimum image reconstruction. The further orientations or directions from which also X-ray projection images of the object to be imaged must be made, can be calculated on the basis of the already recorded projection images.

The present invention hence proposes a system in which, based on the data of a sensor system for measuring a reference direction (e.g. gravity sensor), the determination of spatial properties of recorded X-ray images, detection of the imaging properties of the X-ray unit and the reconstruction of volume image data and also user guidance can be implemented.

A particular advantage of this system is the possibility of simple integration of this 3D imaging function (in software and/or hardware) in present X-ray units (in particular C-arm X-ray systems) without requiring to undertake mechanical changes.

The position sensor is thereby rigidly connected to the recording unit (i.e. the unit comprising X-ray tube and detector). By reading out the sensor measuring values, the orientation of the X-ray tube and of the X-ray detector relative to the predefined reference direction (gravitational direction) can be determined. Since any change in orientation of the X-ray recording system causes a measurable change in the direction data, position information can be assigned to the direction data. The parameters of the transformation or assignment specification required for this purpose can be determined by a calibration process. After the spatial position for each X-ray projection image is determined, finally the volume reconstruction or the tomographic image reconstruction can then be implemented. Furthermore, it is possible to calculate and display instructions for optimal use of the system.

Preferably, the system according to the invention has the position sensor for measuring the reference direction relative to the X-ray recording unit, a computer for converting direction data or orientation data into position data, a reconstruction unit for calculating volume image data from the projection images and the projection data and also a user interface for displaying image data and for interaction with the user. The sensor unit thereby preferably measures the direction of gravitational acceleration and is rigidly integrated in the X-ray unit or disposed thereon. Furthermore, the software preferably generates information relating to operation and orientation of the X-ray unit with the aim of recording image data in the optimal image position which is optimal for the reconstruction.

In order to measure horizontal and vertical movements which have no influence on the orientation of the system relative to the gravitational field, the described position sensor can possibly be used or additional sensors can be used in order to detect such movements. The detection of these additional translator), movements can be effected directly (e.g. with distance-, position sensors) or via evaluation of acceleration data (double integration of the acceleration over time produces the path covered).

In addition to the advantages described already above, the present invention, relative to the systems known from the state of the art, have above all the following advantages:
A simple concept for retrofitting existing X-ray recording systems with a 3D function is made available.
No mechanical changes to the unit itself are thereby required.
The system leads to no restrictions in the movement possibilities of the recording unit by the above-described extension.
All movement axes can hence be used for the recording of projection image data which can be used for the reconstruction.

Subsequently, the invention is now described with reference to a detailed embodiment.

### DETAILED DESCRIPTION OF THE ILLUSTRATED EMBODIMENTS

There are thereby shown:

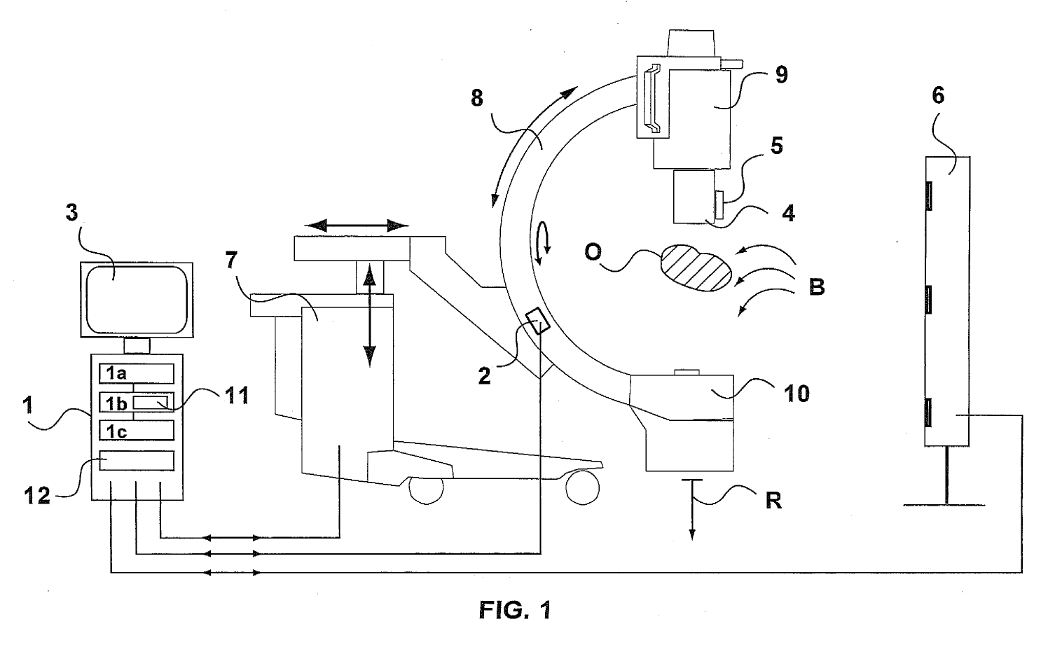

FIG. 1 the basic configuration of the X-ray image recording system according to this embodiment;

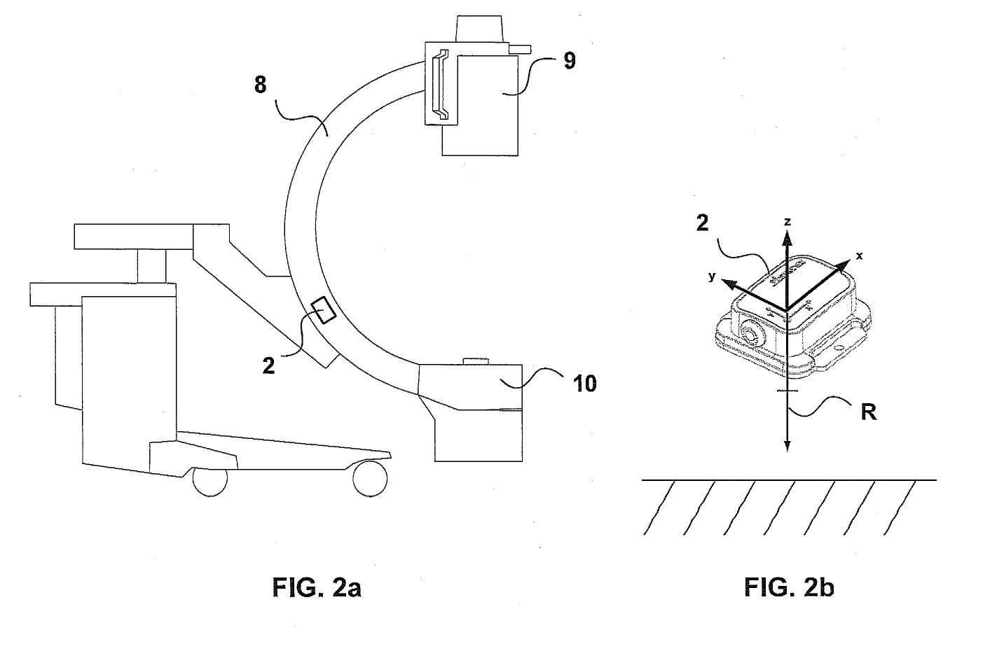

FIGS. 2a-2b the application and principle of use of the position sensor which is used;

FIG. 3 the individual movement axes of the recording system, given by way of example;

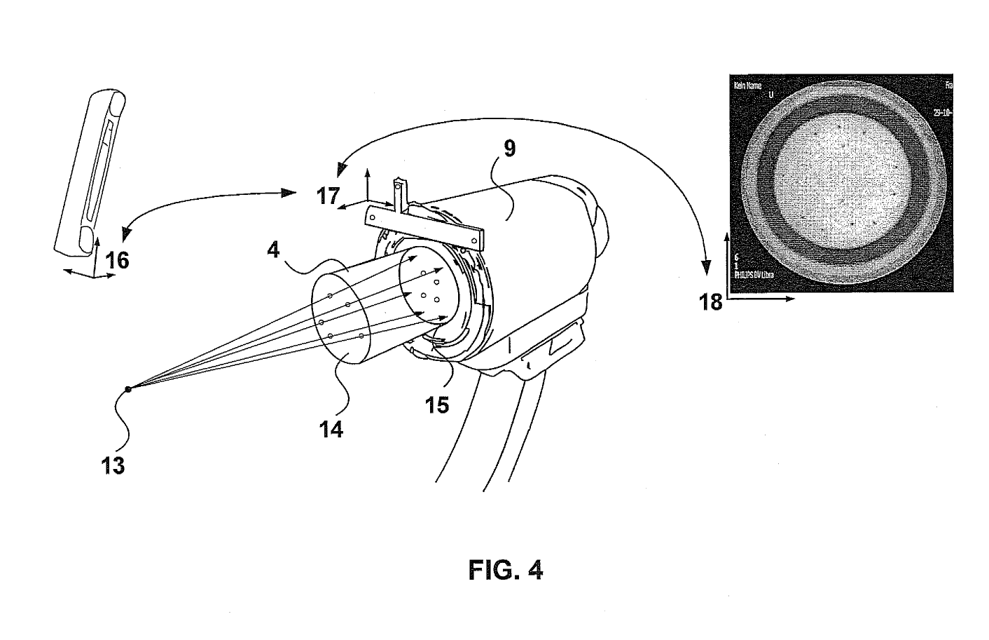

FIG. 4 the principle of calibration of the recording system according to FIG. 1;

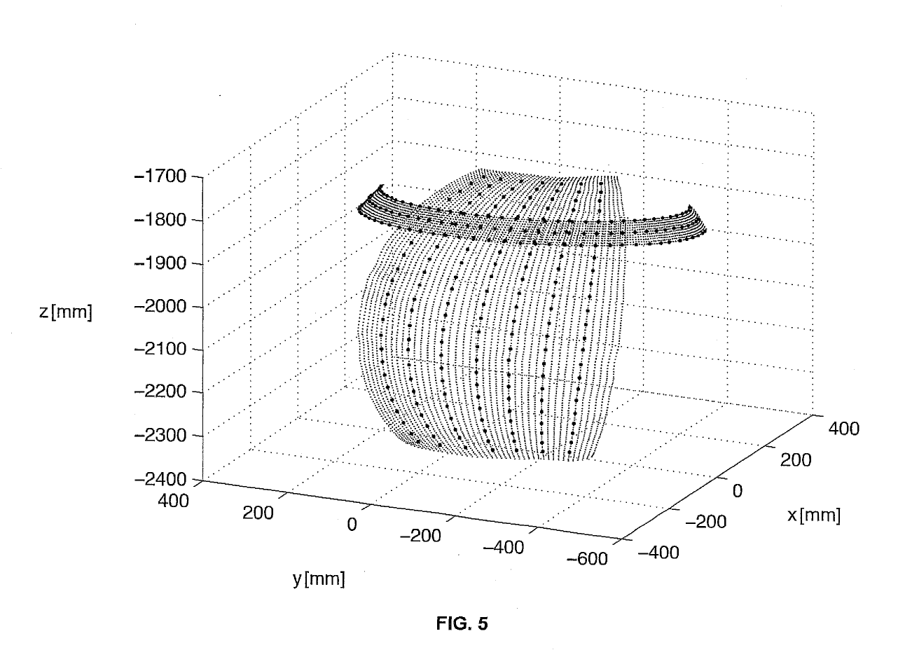

FIG. 5 support points determined during a calibration and support points of a calibration table (LUT), given by way of example and interpolated therefrom;

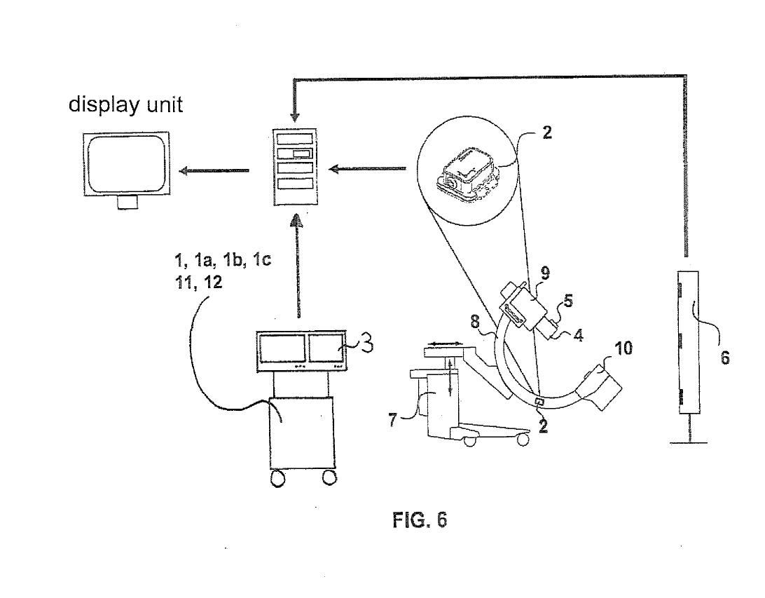

FIG. 6 the system components of the system of FIG. 1 during calibration;

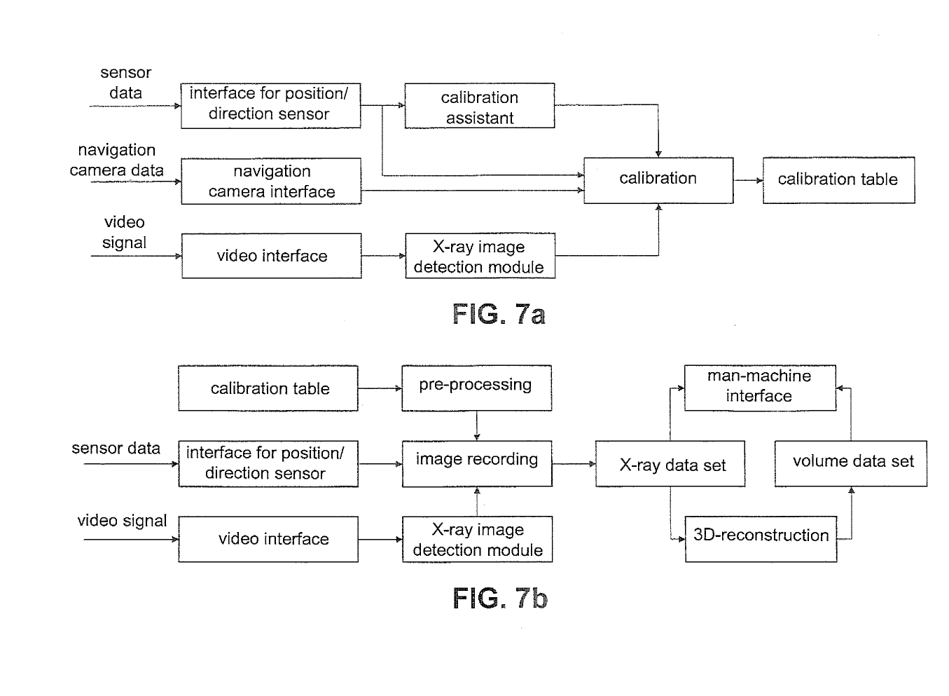

FIGS. 7a-7b the data flow of the software components of the example system of FIG. 1 during calibration and during the reconstruction phase.

FIG. 1 shows an X-ray image recording system according to the present invention in a first embodiment. The X-ray image recording system is constructed on the basis of a standard C-arm. The C-arm 8, at its first end, carries an X-ray image detector 9 (here an analogue detector in the form of an X-ray image amplifier BV, however it can also concern a digital flat image detector) and, on its second opposite end, the X-ray tube 10. In the centre of the C or between the two ends of the C there is situated, between the X-ray tube 10 and the X-ray detector 9, the imaging region B in which the object O (e.g. patient) to be imaged is disposed in the optical path of the X-ray tube and in the image region detected by the detector. As indicated by various arrows (see also FIG. 3), the recording system which comprises the C-arm 8, the X-ray tube 10 and the X-ray detector 9 can be rotated about two axes which are orthogonal relative to each other, the C-axis 19 and the P-axis 20 (cf. FIG. 3). The rotation about the P-axis hereby allows a rotation of the tube 10 and of the detector 9 out of the image plane or perpendicular to the image plane, the rotation about the C-axis 19 (which is perpendicular to the image plane) hereby allows a rotation of these components in the image plane.

Due to the C-arm 8, the X-ray detector 9 is disposed at a fixed spacing and in a fixed position relative to the X-ray tube 10. The spacing and the relative position of the X-ray detector 9 relative to the X-ray tube 10 hence is maintained even during corresponding rotational movements. The further translatory movements of the recording system 8 to 10 in the direction of the P-axis 20 and perpendicular to the P-axis and to the C-axis 19 are possible by means of the lifting axis 21 and the thrust axis 22 (cf. FIG. 3).

A position sensor 2 in the form of a gravity sensor is now disposed connected in a fixed manner to the C-arm 8 on the latter externally. As is described subsequently in even more detail, there can be determined with this position sensor 2 for each momentary position of the system, X-ray tube-X-ray detector, in space, the orientation of this position relative to the pre-defined reference direction R. The pre-defined reference direction R is here the direction of gravitational acceleration or the gravitational vector.

Furthermore, the C-arm device unit 7 supporting the actual C-arm is shown in the picture. This is connected for signal transmission to a central computer 1 (which can comprise for example a PC). The data or momentary orientations of the X-ray tube and of the X-ray detector detected by the position sensor 2 are transmitted via data connection lines to the central computer. Here the data exchange can be configured bidirectionally so that, on the part of the central computer 1, the corresponding sensor functionalities of the position sensor 2 can be adjusted or changed.

The central computer comprises a memory unit 1a (here: hard disc), a computer 1b (here: CPU and main memory of a PC) with a conversion unit 11 disposed therein in the form of a look-up table LUT and also a reconstruction unit 1c (here: separate reconstruction PC) and an instruction unit 12, the function of which is described subsequently in more detail. The individual units 1a, 1b, 1c and 12 are connected to each other for data exchange. The individual units can hereby be produced in the form of hardware units (e.g. memory or the like) and/or in the form of software components (programmes or data structures).

A display unit 3 (monitor or the like) is connected to the central computer 1, with which display unit recorded X-ray projection images or also the reconstructed tomographic images can be displayed.

Finally, the Figure also shows a calibration unit 4 to 6 which, in the present case, comprises a position measuring system 6 in the form of a position camera and a calibration body 4 with markers 5. These elements 4 to 6 are present merely during calibration of the presented unit and are removed before the actual recording operation or patient operation. The markers 5 are the markers, the position of which is detected by the position measuring system 6. These can be for example reflecting spheres, LEDs or the like.

Therefore associated with the structural components of the illustrated recording system are a control computer 1 with incorporated video digitalising card and software, a position or acceleration sensor 2 and a display system 3. The calibration body 4 with the markers 5 is used for calibration for an external position measuring system 6. The video output of the C-arm assembly 7 which is used is connected to the video digitalising card of the control computer 1. The position sensor 2 is mounted on the C-arm unit 8 as described so that a rigid connection between position sensor 2 and X-ray image receiver 9 and X-ray source 10 is produced. The data output of the position sensor 2 is connected to the control computer 1. The display of the data is then effected on the display unit 3. For calibration of the system, the calibration body 4 is fitted on the X-ray detector 9 and the position measuring system 6 is connected to the control computer 1.

As described above already, the calibration operation of the illustrated X-ray image recording system is effected as follows: for a large number of different positions of the X-ray tube-X-ray detector 9, 10 system in space, the position of the X-ray detector 9 and of the X-ray tube 10 in space is detected with the help of the calibration unit 4 to 6. For this purpose, the calibration body 4 is connected rigidly to the X-ray detector 9. The calibration body 4 concerns a body of fixed three-dimensional geometry, from the detection of which with the camera system 6 and parallel recording and evaluation of an X-ray image the relative position of the tube-detector system 9, 10 in space can be determined unequivocally. From the detected and evaluated X-ray image- and position data, all those position data with respect to the position of the system 9, 10 in space are determined, evaluated and stored in the memory unit 1a, which data are required in order to be able to use an X-ray projection image which is recorded in this position for reconstruction of tomographic images.

During the calibration, the position data required for the reconstruction are determined completely. By implementing the calibration at a large number of different positions, also position-dependent influences on the X-ray system, e.g. deformation of the mechanical unit due to the high intrinsic weight, are imaged.

Storage of these required position data is effected together with the associated orientation data (which were determined by the position sensor 2 in the same position of the system 9, 10) in the memory unit 1a. If position data and associated orientations at a sufficient number of support points or at a sufficient of different positions of the system 9, 10 in space have been detected and stored, then a look-up table LUT 11 is generated from these data with the help of the computer 1b, which table allows conversion or transformation between orientation data and associated required position data. The orientations and required position data stored together are subsequently also termed calibration data.

During operation of the system the calibration data are hence recorded initially, which data are required in order to be able to determine the position of the X-ray source in space (or the position of the system comprising X-ray tube and X-ray detector 9 and 10) in the subsequent examination operation for each X-ray projection image. For this purpose, the calibration body 4 is fitted on the image amplifier and the position thereof is measured continuously at a sufficient number of support points. The calibration body 4 here consists of a three-dimensional geometry which is also visible in the X-ray image and serves for determining the imaging properties of the X-ray system with the help of the position measuring system 6. Whenever the recording of a new X-ray projection image is established, the position measuring system 6 determines the spatial position of the calibration body 4. With the help of the predetermined geometry of the calibration body 4, the imaging thereof in the detected X-ray image and the position data detected by the measuring system are determined, then the position of the X-ray projection image or the position of the X-ray tube 10 and of the detector 9 and the projection properties of the C-arm are determined and stored together with the gravitational acceleration values of the position sensor 2 as calibration data. This process is repeated at a sufficiently large number of support points or positions of the tube-detector system 9, 10 in space. As a function of the system state and the sensor data of the position sensor 2, as subsequently described in even more detail, user instructions are furthermore generated by the instruction unit 12, which instructions assist the user in the system calibration or convey to him the required information with respect to at which further support points calibration data should still be detected.

In the actual recording operation or patient operation, the elements 4 to 6 which are required merely for the previously described calibration operation are removed. The computer system 1 or the memory unit 1a and computer 1b thereof are now configured such that after recording an X-ray projection image (at a defined position of the tube-detector system 9, 10 in space) with reference to the thereby detected sensor values of the position sensor 2 (orientation data relative to the reference direction or gravitational direction R), those position data of the X-ray projection image which are required for use thereof for the image reconstruction of tomographic images can be calculated from the stored calibration data. For this purpose, the above-described look-up table is used: by means of this the orientation relative to the reference direction is transformed into the associated position data. This can take place for example with the help of a spline interpolation method, as is known to the person skilled in the art, with the help of which the required position data of the recorded X-ray projection image are determined from the support point orientations of the calibration data which are closest to the orientation of the recorded X-ray projection image.

If X-ray projection images of the object O were recorded in the imaging region B from a sufficient number of different spatial directions (for example over a periphery of 180°+fan angle of the X-ray beam fan of the X-ray source detected by the detector), then, from these recorded images with the help of the required position data interpolated from them and from their orientations with the help of the LUT, the desired tomographic images of the object O can be reconstructed with the help of the reconstruction unit 1c of the computer system 1.

Within the scope of the recording or patient operation, the instruction unit 12 of the computer system 1 is used for the purpose of establishing from which spatial directions or with which positions of the tube-detector system 9, 10 for the chosen reconstruction algorithm, also further X-ray projection images should be recorded for optimisation of the image quality of the reconstruction images. The instruction unit 12 gives the operator corresponding instructions then by means of a display on the monitor 3. Calculation of the further required projection directions thereby takes place on the basis of the calculated position data of the already recorded X-ray projection images.

Hence during application of the system for 3D image recording during patient operation, the software/hardware of the control computer examines the video input and detects with reference to the change in the image content the recording of a new X-ray projection image. If the control computer 1 detects the recording of such a new X-ray projection image, the values of the position sensor 2 for this point in time are stored. In the recorded calibration data, then position information or position data with similar sensor data (i.e. with a similar position of the X-ray detector system 9, 10) are sought. This takes place with the help of suitable interpolation methods. With these interpolation methods, the position of the recorded X-ray projection image and the position of the recording X-ray source are determined. The spatially assigned projection images are stored in the system. Finally a 3D reconstruction is calculated with the help of reconstruction algorithms, known to the person skilled in the art, from the recorded projection images, i.e. a corresponding data set of 3D tomographic images. The projection images, the data set of 3D tomographic images and the spatial correlations are displayed for the user. As a function of the system state, the already recorded X-ray projection images and the detected sensor data of the position sensor 2, user instructions are generated via the unit 12 and assist the user in the operation of the system, in particular in the orientation of the recording unit for projection directions still to be recorded.

Further properties of the X-ray image recording system according to the invention, which are described in the above embodiment, are now described.

### Dependence Between C-Arm Position and the Direction of Gravitational Acceleration:

A concrete implementation of the invention consists of a C-arm 8 and an acceleration sensor 2. The sensor is connected rigidly to the C-structure and hence immovably relative to the image amplifier and the X-ray source (FIG. 2). During use, the direction of gravitational acceleration is measured with the help of the acceleration sensor. The measuring value is present in the form of a vector in the internal coordinate system of the sensor (see FIG. 2). A change in orientation of the sensor, from the point of view of the internal coordinate system, causes a change in direction of the vector as long as the axis of rotation is not parallel to the acceleration vector. In the case of the drawing, a rotation of the sensor about the z-axis therefore causes no change in the gravitational acceleration vector.

FIG. 2a shows the C-arm with mounted sensor 2. FIG. 2b shows the internal coordinate system of the acceleration sensor 2 with a vector, given by way of example, for gravitational acceleration. A rotation of the sensor about the axis of the gravitational acceleration vector does not have an effect on the direction of the vector in the reference system.

FIG. 3 shows schematically the construction of a C-arm 8, including the typical joints. The X-ray source 10 and the detector 9 (image amplifier) are situated on a C-shaped structure. By means of rotation of the C-structure about the C-axis 19 or P-axis 20, X-ray images of an object can be recorded from any directions. The image amplifier 9, the X-ray source 10 and the acceleration sensor 2 thereby are moved on a convex surface (can be assumed in the model to be a sphere). Any movement of the C-structure thereby corresponds to a rotation of the sensor about the C- or P-axis. As long as this axis of rotation is not parallel to the gravitational acceleration vector, the various C-arm positions can be differentiated unequivocally from each other by means of the gravitational acceleration direction.

More extensive movements of the C-structures are possible by using the lifting and thrust axis, and also by a movement of the moveable stand. These movements do not change the orientation of the sensor in space and cause no change in gravitational acceleration in the internal coordinate system. Nevertheless it is theoretically possible that the accelerations which occur during such movements are measured and used for calculating the movement path.

### Mode of Operation of the Calibration:

It is the aim of the calibration to determine the position of the X-ray image and the position relative to a basic coordinate system BKS 16. This BKS 16 is defined in the simplest case by the optical measuring system which is used for the calibration. FIG. 4 shows the two-stage calibration process for a C-arm position. The two stages are described in the following:

### 1. Determination of the Position of the Image Plane:

In a plane (recording plane 15) close to the image amplifier 9, lead markers are applied at positions defined in the reference coordinate system. Detection of the marker shadows in the X-ray image (1 mg) 18 enables determination of the image location and position relative to the reference coordinate system and consequently the transformation ^{BKS}T_{Img} by means of point-to-point matching. This is possible since the positions of the lead markers in the coordinate system CalBody 17 are known from the sublayers of the construction and the transition between CalBody 17 and BKS 16 is measured by the optical measuring system.

### 2. Determination of the Position of the X-Ray Source:

In a second plane (calibration plane 14), lead markers are likewise applied at known positions. The marker shadows are detected in the image and converted into 3D positions with the help of the transformation ^{BKS}T_{Img} known from step 1. As a result, the projection beams for the lead markers of the calibration plane can be calculated. At the intersection point of these beams there is situated the X-ray source 13.

FIG. 4 hence shows the calibration of a C-arm by determining the position of the image and the X-ray source 9 relative to the basic coordinate system BKS 16. The result of this calibration is the position of the image in the BKS, including the scaling parameter (dimension of the image points). This calibration process is implemented for various positions of the C so that the entire rotational range is covered. For each position, the current gravitational acceleration vector and the two transformations are stored in a table.

Derivation of the Position Data from the Gravitational Data During Use:

During the actual system use, the system detects the recording of a new X-ray image, e.g. by continuous analysis of the video signal. If a new X-ray image is present, acceleration data of a defined time window are stored together with the image data. By analysing the scattering of the acceleration values during the image recording time window, it can be checked whether the C-arm was stationary during the image recording. The inputs which are closest to the measured gravitational acceleration vector are loaded from the calibration table. By interpolation e.g. by means of cubic splines, the position data for the recorded X-ray image can be determined. FIG. 5 shows a 3D view with calibrated and interpolated support points.

### User Instructions:

When using the system, the user must record X-ray images from various directions in order that volume data can be reconstructed from the projection images. The reconstruction quality thereby increases with the number of images and the angle range scanned. In order to improve the reconstruction quality in a targeted and efficient manner, it is sensible to generate user instructions with the instruction unit 12, which assist the user in the orientation of the C-arm. It can be calculated with reference to the position data of the already recorded images from which position further images should be recorded in order to improve the reconstruction quality as effectively as possible.

Such user instructions likewise help in the orientation of the C-arm towards the patient.

### Function Description:

The 3D imaging system according to the invention extends standard C-arms by the 3D imaging functionality. For this purpose, for example when using an image amplifier as detector, a position sensor is fitted on the C-arm and the video image is tapped from the video output. The C-arm is therefore neither changed in construction nor is it restricted in its functionality. The system has to be calibrated once by an engineer with the help of a position camera. The doctor can record images as usual and observe these. In addition, a current reconstruction result is available to him at any time. This can be observed by the doctor in the usual tomographic view. In order to ensure an optimal reconstruction result, ideal recording positions are recommended to the doctor by the instruction unit 12.

By means of the above-mentioned characteristics, the system enables economical and flexible 3D visualisation for pre-, intra- and post-operative use.

Important components of the 3D C-arm imaging system are thereby
1. computer,
2. screen for visualisation of the reconstructed volume and the recorded X-ray images,
3. position sensor for determining the C-arm orientation in space,
4. input devices, such as mouse and keyboard,
5. equipment for the C-arm calibration: calibration body including tracker and navigation camera.

It is the function of the system to produce 3D image data from 2D X-ray images from standard C-arms and to display these. The 2D data are tapped directly from the C-arm for example as video signal, digitalised and analysed. The mode of operation of the C-arm is not restricted. The system has a separate voltage supply connection and is furthermore operated for example at the analogue video output of a C-arm.

After the system has been connected to the C-arm and switched on, the application starts automatically. Firstly the desired recording strategy (image recording along the propeller axis or P-axis or the C-axis) must be selected. The chosen recording strategy influences both the C-arm positions at which images must be recorded and the type of the following dialogue for orientating the C-arm. For control of the orientation, the current X-ray image is displayed. The user must position the object in the centre of the image at two different angle positions. A crosshair which assists with centering of the object to be reconstructed is superimposed in the video image as positional assistance. Subsequently, the man-machine interface is started by the recording assistant.

The recording assistant 12 assists the user in the recording of the X-ray images. The C-arm positions to be approached, at which respectively an image must be made, are conveyed to him. The reconstruction, the image detection and the volume display operate independently of each other so that X-ray images can be recorded even during a current reconstruction.

The man-machine interface makes it possible for the user to view the current reconstruction result at any time. The volume is visualised in axial, coronal and sagittal tomographic view. The recorded X-ray images are displayed in a further window. With the forward and backward button, the X-ray images can be seen clearly, or can be switched to the volume view with the mode button. It is possible to zoom into all the views and also to switch separately to full image mode.

### Structure of the System Components:

Belonging to the structural elements of the example system are a PC 1 with incorporated video digitalisation card, an acceleration sensor 2, a navigation system 6, a display unit 3 and a calibration body 4. The components are connected to each other electrically and mechanically as follows (FIG. 6). The video output (BNC) of the mobile viewing station is connected to the video digitalisation card incorporated in the PC. The acceleration sensor is mounted (screwed or glued) onto the C of the C-arm and connected by the adaptor cable to the PC. For visualisation of the data, the jointly delivered display unit is connected to the PC. During calibration of the system, the following components are connected to the system. The calibration body is fitted on the image amplifier (screwed or glued), the tracker requiring to point towards the open side of the C. The navigation system is connected likewise to the PC via a serial cable and positioned on the front side towards the C-arm.

Dynamic Behaviour of the Software During the Calibration (FIG. 7a):

After the software has been started in calibration mode, the system is tested for functional capacity of the components required for the calibration. After determination of the sensor position relative to the C-arm (by means of two defined C-arm positions), the X-ray image detection module is activated and the digitalised video image is tested for new X-ray images.

The user approaches, with the C-arm, the positions displayed by the calibration assistant, carries out an X-ray recording respectively at these places and waits respectively for a positive response of the system.

As soon as a new X-ray image is detected and this is situated at the output in a stable manner over a specific time, the image is supplied for calibration. The calibration detects the markers in the inner image region and calculates the position of the image plane relative to the BV tracker therefrom. With the help of the external markers and projections thereof in the image, the position of the X-ray source relative to the image centre is determined. In order to suppress image interference which is produced during the digitalisation, 19 additional video images can be recorded and calibrated individually. The median of the 20 determined image parameters is calculated. The determined parameters and the position and location data of the calibration body are stored respectively with the current position data respectively in a calibration table.

### Description of the Software Components During Calibration of the C-Arm:

| | |
|---|---|
| Navigation | Makes available to the system the position and the |
| camera | orientation of the calibration body in the navigation camera |
| interface: | system. The data are averaged over 100 values in order to |
| | suppress noise. In addition, a movement monitoring takes |
| | place. |
| Acceleration | Communication with the acceleration sensor. In addition, |
| sensor | the acceleration values in X, Y and Z direction are buffered |
| interface: | and can be called up, when averaged, over an arbitrary |
| | period of time (maximum buffer length). An analysis |
| | function enables interference detection over the required |
| | averaging period of time. |
| Video | Interface for video digitalisation card. It makes the current |
| interface: | video image available to the system. |
| X-ray image | Examines the video image cyclically with the help of a |
| detection | differential image method for differences in order thus to |
| module: | detect new X-ray images. Only specific regions are |
| | monitored taking into account the temporal and image |
| | properties of the C-arm. If a threshold is exceeded, the |
| | current video image is supplied to the image calibration |
| | module as new image. |
| Calibration | The man-machine interface displays the next position to be |
| assistant: | approached and the angle difference to be covered. |
| Calibration: | Produces the current calibration data set from the sensor-, |
| | navigation- and geometric data, obtained from the X-ray |
| | image. This consists of the position of the calibration body |
| | in space, the image position relative to the calibration body |
| | and also the relative position of the X-ray source. |
| Calibration | The position of the calibration body in space, the image |
| table: | position relative to the calibration body and also the relative |
| | position of the X-ray source are stored in separate data files. |

Dynamic Behaviour of the Software During Operation (FIG. 7b):

After the programme start, the user informs the system as to which recording strategy he would like to use. For this purpose, a recording strategy selection dialogue is indicated, which loads the corresponding calibration tables according to the selection and subsequently issues specific C-arm orientation instructions to the user. In order to assist the user, the current video image is given.

The loaded calibration tables firstly pass through pre-processing. New support points are hereby extrapolated and new values are interpolated between all the support points. Subsequently, the X-ray image detection module is activated.

The user guide displays the next C-arm position to be approached visually. The X-ray image detection periodically checks the digitalised video signal from the analogue video output of the C-arm. As soon as a new X-ray image is detected and this is present in a stable manner at the output over a certain time, it is accepted into the system as new X-ray image and, together with the averaged position data, is supplied for image recording. This comprises a brightness correction and also masking and inversion of the image. With reference to the position data of the sensor, closely situated support points are sought and interpolated linearly between these. The thus obtained position data are allocated to the image and stored. Subsequently, the image is displayed as new X-ray image in the man-machine interface and added to the X-ray image reconstruction list. The system now jumps back to the video monitoring mode and is ready for new X-ray images.

The reconstruction algorithm establishes whether new X-ray images are present and, if necessary, starts a new reconstruction over all the images. The current progress is displayed in a progress bar. When the reconstruction has been implemented, the new volume is loaded into the man-machine interface and the contrast is automatically regulated. The 3D reconstruction algorithm operates independently of the X-ray image detection and the image recording such that the system can record new X-ray images whilst the current reconstruction has not yet concluded. In addition, the result of the last reconstruction and all the recorded X-ray images can be observed in parallel with the man-machine interface.

The man-machine interface makes it possible for the user to view the current reconstruction result at any time. The volume is visualised in axial, coronal and sagittal tomographic view. It is possible to zoom in on these and also to switch separately to full image model. The recorded X-ray images are displayed in a further window. With the forward and backward button, the X-ray images can be viewed clearly, or can be switched to the volume view with the mode button. The full image mode is also available for this window.

Description of the Software Components During the Reconstruction Operation (FIG. 7b):

| | |
|---|---|
| Video | Interface for the video digitalisation card. It makes the |
| interface: | current video image available to the system. |
| Acceleration | Communication with the acceleration sensor. In addition, |
| sensor | the acceleration values in X, Y and Z direction are buffered |
| interface: | and can be called up, when averaged, over an arbitrary |
| | period of time (maximum buffer length). An analysis |
| | function enables a movement detection over the required |
| | averaging period of time. |
| X-ray image | Examines the video image cyclically with the help of a |
| detection | differential image method for differences in order thus to |
| module: | detect new X-ray images. Only specific regions are |
| | monitored taking into account the temporal and image |
| | properties of the C-arm. If a threshold is exceeded, the |
| | current video image is supplied to the image recording |
| | module as new image. |
| Calibration | Contains the assignment tables of the imaging properties |
| table | with respect to the values of the acceleration sensor. |
| (LUT): | |
| Pre- | Extrapolates additional support points from the loaded |
| processing: | calibration tables and interpolates support points at a 1° |
| | spacing. |
| Image | Subjects the X-ray image to pre-processing and, with |
| recording: | reference to the current acceleration values, the closest |
| | support points are determined and the corresponding image |
| | imaging parameters are interpolated linearly between them |
| | and allocated to the image. |
| X-ray image | List of all the previously recorded X-ray images. |
| data set: | |
| 3D re- | Starts a new 3D volume reconstruction if new pictures are |
| construction: | present and the present reconstruction has been concluded, |
| | Furthermore, the contrast parameters of the volume are |
| | determined for the MMS. |
| Volume | Contains the currently finished reconstructed volume. |
| data set: | |
| Man- | Displays the current volume data set in tomographic view, |
| machine | and also the previously recorded X-ray images or 3D views |
| interfaces: | of the layers. |

### EXPLANATION OF THE FIGURES

central computer having:
1a memory unit
1b computing unit
1c reconstruction unit
2 position sensor
3 display unit
4 calibration body
5 markers
6 position measuring system
7 C-arm assembly
8 C-arm unit
9 X-ray image detector
10 X-ray tube
11 conversion unit
12 instruction unit
13 X-ray source position
14 calibration plane of the calibration body
15 recording plane of the calibration body
16 basic coordinate system BKS
17 calibration body coordinate system CalBody
18 image coordinate system img
19 C-axis
20 P-axis
21 lifting axis
22 thrust axis
B imaging region
O object to be imaged
R reference direction

1. An X-ray image recording system for recording X-ray projection images and orientation information for recorded X-ray projection images, comprising:
an X-ray tube and an X-ray image detector disposed in the optical path of the X-ray tube for recording X-ray projection images of an object to be imaged, which can be disposed and/or is disposed in a fixed manner between the X-ray tube and the X-ray detector in an imaging region, the X-ray tube and the X-ray detector being disposed in a fixed manner relative to each other and being moveable around the imaging region at least in a sector,
a position sensor which is disposed in a fixed manner relative to the X-ray tube and to the X-ray detector and with which, at the moment of recording of an X-ray projection image, the momentary orientation of the X-ray tube and of the X-ray detector relative to a pre-defined reference direction can be determined, and
a memory unit for storing recorded X-ray projection images together with the respectively associated momentary orientation of the X-ray tube and of the X-ray detector.

2. The X-ray image recording system according to the claim 1,
wherein
the position sensor is an acceleration sensor, in particular a gravity sensor, with which the momentary orientation of the X-ray tube and of the X-ray detector can be determined relative to a predefined acceleration direction as the reference direction, in particular relative to the direction of gravitational acceleration.

3. The X-ray image recording system according to claim 1,
wherein
a computer which is connected to the memory unit for data exchange and with which, for the purpose of a tomographic image reconstruction which is based on a plurality of recorded X-ray projection images, for each recorded X-ray projection image used for the reconstruction, those position data of the X-ray projection image, which are required for calculation of the tomographic images of the reconstruction, can be calculated from the stored, associated momentary orientation.

4. The X-ray image recording system according to claim 1,
wherein
the memory unit and/or the computer includes a predetermined conversion unit, in particular a conversion table preferably present in the form of a look-up table (LUT), for converting an orientation relative to the reference direction into the position data associated with this orientation and required for calculating the tomographic images of the reconstruction.

5. The X-ray image recording system according to claim 4,
wherein
a reconstruction unit which is connected to the memory unit and/or to the computer for data exchange and with which, from stored X-ray projection images and the associated position data, a tomographic image reconstruction can be performed.

6. The X-ray image recording system according to one claim 4,
wherein
the conversion unit can be preset and/or is preset by means of a calibration unit.

7. The X-ray image recording system according to claim 6,
wherein
the calibration unit has a calibration body which is disposed in the optical path of the X-ray tube which can be detected and/or is detected by the X-ray detector, and which is disposed in a fixed manner relative to the X-ray tube, to the X-ray detector or to the X-ray tube and to the X-ray detector.

8. The X-ray image recording system according to claim 7,
wherein
the calibration unit has a position measuring system, in particular a position camera and/or a navigation system camera,
with which the spatial position and/or orientation of the calibration body can be scanned for determining position data which are required for calculation of the tomographic images of a tomographic image reconstruction, and
with which these position data, which are obtained from scanning the calibration body in a defined spatial position and/or orientation, together with the momentary orientation of the X-ray tube and of the X-ray detector relative to the predefined reference direction, which is determined by means of the position sensor at the moment of this scanning, can be stored as calibration data and/or can be transmitted for storage, preferably for storage in the memory unit.

9. The X-ray image recording system according to claim 3,
wherein
the position data required for calculation of the tomographic images of the reconstruction can be obtained, with the computer, from the calibration data with the help of an interpolation method, in particular by means of a spline-based interpolation by using the stored, associated momentary orientations of the X-ray projection images to be used for the reconstruction.

10. The X-ray image recording system according to claim 1,
wherein
precisely one predefined reference direction.

11. The X-ray image recording system according to claim 1,
wherein
an instruction unit with which, on the basis of the associated momentary orientations of already recorded X-ray projection images, further orientations of the X-ray tube and of the X-ray detector can be calculated and indicated, at which further X-ray projection images for use for a subsequent tomographic image reconstruction should be recorded.

12. The X-ray image recording system according to claim 3,
wherein
the further orientations can be calculated from calculated position data of already recorded X-ray projection images.

13. The X-ray image recording system according to claim 1,
wherein
a display unit for displaying recorded X-ray projection images and/or tomographic images of a tomographic image reconstruction performed on the basis of recorded X-ray projection images.

14. The X-ray image recording system according to claim 1,
wherein
a C-arm unit, in particular a C-arm unit the C-arm of which can be rotated about two axes (C-axis and P-axis) which are orientated preferably orthogonally relative to each other, on the one C-arm end of which the X-ray tube and on the other C-arm end of which the X-ray detector is fixed.

15. The X-ray image recording system according to claim 14,
wherein
the position sensor is fixed on the C-arm such that a rigid connection between the position sensor, the X-ray tube and the X-ray detector is formed.

16. An X-ray image recording method for recording X-ray projection images and orientation information for recorded X-ray projection images,
an X-ray tube and an X-ray image detector, placed in the optical path of the X-ray tube, for recording X-ray projection images of an object to be imaged, which object has been disposed in a fixed manner between the X-ray tube and the X-ray detector in an imaging region (B), being disposed in a fixed manner relative to each other and being moved around the imaging region at least in a sector,
a position sensor being disposed in a fixed manner relative to the X-ray tube and to the X-ray detector,
the momentary orientation of the X-ray tube and of the X-ray detector relative to a predefined reference direction being determined with the position sensor at the moment of the recording of an X-ray projection image, and
the recorded X-ray projection images together with the respectively associated momentary orientation of the X-ray tube and of the X-ray detector being stored.

17. The X-ray image recording method according to claim 16,
wherein
an X-ray image recording system is used for the recording.
